(19) United States
(12) Patent Application Publication
(10) Pub. No.: US 2005/0273092 A1
(43) Pub. Date: Dec. 8, 2005

(54) Method and apparatus for shrinking tissue

(76) Inventors: Antonio Mendez G., Mexicali (MX); Steve Khalaj, Laguna Hills, CA (US); Moises Valle, Tustin, CA (US)

Correspondence Address: IRELL & MANELLA LLP, 840 NEWPORT CENTER DRIVE, SUITE 400, NEWPORT BEACH, CA, 92660, US

(21) Appl. No.: 10/860,529

(22) Filed: Jun. 2, 2004

(51) Int. Cl. A61B 18/14

(52) U.S. Cl. 606/41

(57) ABSTRACT

An apparatus and method for treating superficial skin conditions with radio frequency energy. The apparatus includes a probe that is coupled to a radio frequency console. The probe has at least one tip that can be inserted into skin tissue. Radio frequency energy can be delivered to the skin tissue through the tip. The radio frequency energy denatures tissue to cause tissue shrinkage. The tissue shrinkage can reduce or remove wrinkles, or reduce or eliminate the visual appearance of blood carrying tissue such as facial spider veins.

### BACKGROUND OF THE INVENTION

1. Field of the Invention

The present invention relates to a method and apparatus for treating non-ocular tissue.

2. Prior Art

Radio frequency energy has been used in various medical procedures. For example, radio frequency probes have been used to cauterize and coagulate blood. Radio frequency energy has also been used in ophthalmic procedures.

Refractec, Inc. of Irvine, Calif., the assignee of the present application, has developed a system to correct hyperopia and presbyopia with a probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The circular band of the denatured areas decreases the radius of the eye. The procedure of applying RF energy through a probe tip to denature corneal tissue is taught by Refractec under the service marks CONDUCTIVE KERATOPLASTY and CK.

Thermage, Inc. of Hayward, Calif. has marketed a product that “removes” wrinkles through the application of radio frequency energy. Thermage has also obtained a number of related patents, including U.S. Pat. Nos. 6,470,216; 6,461,378; 6,453,202; 6,438,424; 6,430,446; 6,425,912; 6,413,255; 6,405,090; 6,387,380; 6,381,498; 6,381,497; 6,377,855; 6,377,854; 6,350,276; 6,311,090; 6,241,753; 5,948,011; 5,919,219; 5,871,524; and 5,755,753. To prevent overheating of the outer layer of skin tissue the Thermage product incorporates a cooling system that creates a reverse thermal gradient through the skin. The cooling system requires a heat exchanger, coolant lines, etc. that increase both the costs and complexity of the system.

VNUS Medical, Inc. of San Jose, Calif. markets a product that treats venous diseases through the application of RF energy. VNUS also obtained a number of related patents, including U.S. Pat. Nos. 6,638,273; 6,613,045; 6,401,719; 6,398,780; 6,361,496; 6,322,559; 6,263,248; 6,258,084; 6,237,606; 6,231,507; 6,200,312; 6,179,832; 6,165,172; 6,152,899; 6,149,660; 6,139,527; 6,135,997; 6,071,277; 6,036,687; 6,033,398; 6,033,397; 6,014,589; 5,810,847; 5,730,136; and 5,609,598. The VNUS products are catheters that are inserted into the body. Such products are not practical for application to small veins, such as facial spider veins that may appear on the face of a patient.

It would be desirable to provide an apparatus and method for treating superficial skin conditions that is relatively inexpensive and easy to perform.

### BRIEF SUMMARY OF THE INVENTION

A method and apparatus for reducing the visual appearance of a blood carrying tissue. The method includes placing a probe in contact with a skin tissue and delivering radio frequency energy to the skin tissue. The radio frequency energy denatures the skin tissue and reduces the visual appearance of the blood carrying tissue.

### BRIEF DESCRIPTION OF THE DRAWINGS

FIG. 1 is a perspective view of a system for treating superficial skin conditions;

FIG. 2 is a graph showing a waveform that is provided by a console of the system;

FIG. 3 is an enlarged view of a tip inserted into skin tissue;

FIG. 4 is an alternate embodiment of a probe;

FIG. 5 is an alternate embodiment of a probe inserted into skin tissue;

FIG. 6 is an alternate embodiment of a probe;

FIG. 7 is an alternate embodiment of a probe;

FIG. 8 is an illustration showing a patient with a variety of different ground elements.

### DETAILED DESCRIPTION

Disclosed is an apparatus and method for treating superficial skin conditions with radio frequency energy. The apparatus includes a probe that is coupled to a radio frequency console. The probe has at least one tip that can be inserted into skin tissue. Radio frequency energy can be delivered to the skin tissue through the tip. The radio frequency energy denatures tissue to cause tissue shrinkage. The tissue shrinkage can reduce or remove wrinkles, or reduce or eliminate the visual appearance of blood carrying tissue such as facial spider veins, a condition known as telangiectasia. Inserting the tip into the skin tissue allows for the direct application of radio frequency energy to tissue without creating a thermal tissue gradient that will damage the outer layer of skin. The apparatus therefore does not require a cooling system found in devices of the prior art.

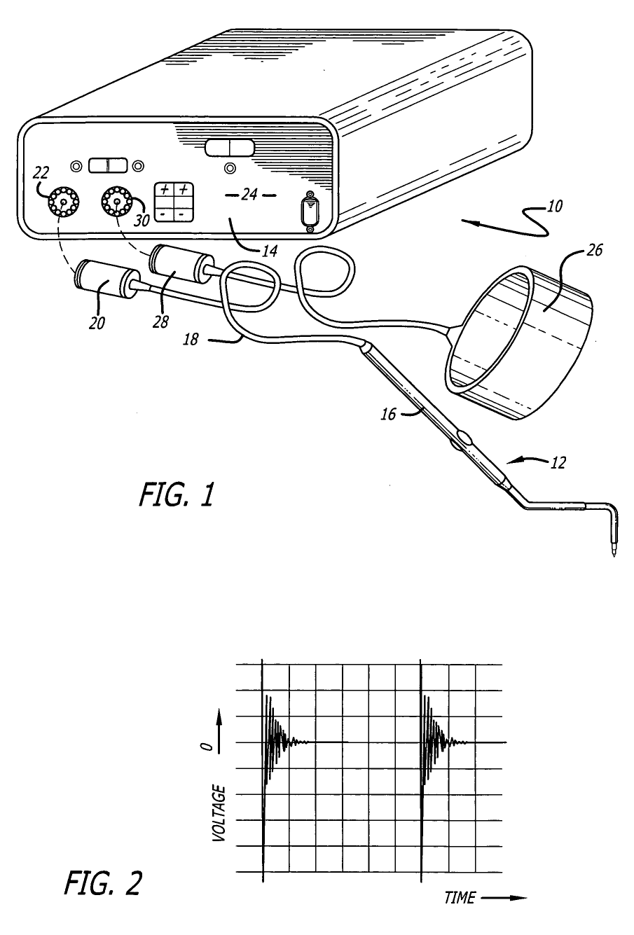

Referring to the drawings more particularly by reference numbers, FIG. 1 shows an embodiment of an apparatus 10 that can be used to treat superficial skin conditions. The apparatus 10 includes an electrode probe 12 coupled to a console 14. The console 14 contains a power supply that can deliver electrical power to the probe 12. The probe 12 has a hand piece 16 and wires 18 that couple the probe electrode to a connector 20 that plugs into a mating receptacle 22 located on the front panel 24 of the console 14. The hand piece 16 may be constructed from a non-conductive material.

The system 10 also includes a return element 26 that is in contact with the patient to provide a return path for the electrical current provided by the console 14 to the probe 12. The return element 26 has a connector 28 that plugs into a mating receptacle 30 located on the front panel 24 of the console 14. By way of example, the return element 26 may be a wrist band as shown, a bar, a pad, a ring, a patch or any other configuration to create a relatively low impedance contact with the patient.

The console 14 may provide a predetermined amount of energy, through a controlled application of power for a predetermined time duration. The console 14 may have manual controls that allow the user to select treatment parameters such as the power and time duration. The console 14 can also be constructed to provide an automated operation. The console 14 may have monitors and feedback systems for measuring physiologic tissue parameters such as tissue impedance, tissue temperature and other parameters, and adjust the output power of the radio frequency amplifier to accomplish the desired results.

In one embodiment, the console 14 provides voltage limiting to prevent arcing. To protect the patient from overvoltage or overpower, the console 14 may have an upper voltage limit and/or upper power limit which terminates power to the probe when the output voltage or power of the unit exceeds a predetermined value.

The console 14 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel 24 of the console 14 typically contains meters and displays that provide an indication of the power, frequency, etc., of the power delivered to the probe.

The console 14 may deliver a radiofrequency (RF) power output in a frequency range of 100 KHz-5 MHz. In the preferred embodiment, power is provided to the probe at a frequency in the range of 350 KHz. The console 14 is designed so that the power supplied to the probe 12 does not exceed a certain upper limit of up to 50 watts. The time duration of each application of power to a particular location of tissue can be up to several seconds.

If the system incorporates temperature sensors, the console 14 could control the power such that the target skin tissue temperature is maintained to no more than 85° C., to avoid necrosis of the tissue. The temperature sensors can be carried by the probe 12, incorporated into the electrodes 40, or attached within proximity to the electrodes 40.

If the system includes an impedance monitor, the power could be adjusted so that the target tissue impedance, assuming a probe 12 with a tip of length 460 um and diameter of 90 um, decreases by approximately 50% from an initial value that is expected to range between 2500 to 4000 ohm. The console 14 could regulate the power down if, after an initial descent, the impedance begins to increase. Controls can be incorporated to terminate RF delivery if the impedance increases by a significant percentage from the baseline. Alternatively, or additionally, the console 14 could modulate the duration of RF delivery such that delivery is terminated only when the impedance exceeds a preset percentage or amount from a baseline value, unless an upper time limit is exceeded. Other time-modulation techniques, such as monitoring the derivative of the impedance, could be employed. Time-modulation could be based on physiologic parameters other than tissue impedance (e.g tissue water content, chemical composition, etc.)

FIG. 2 shows a typical voltage waveform that is delivered by the probe 12 to the skin. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

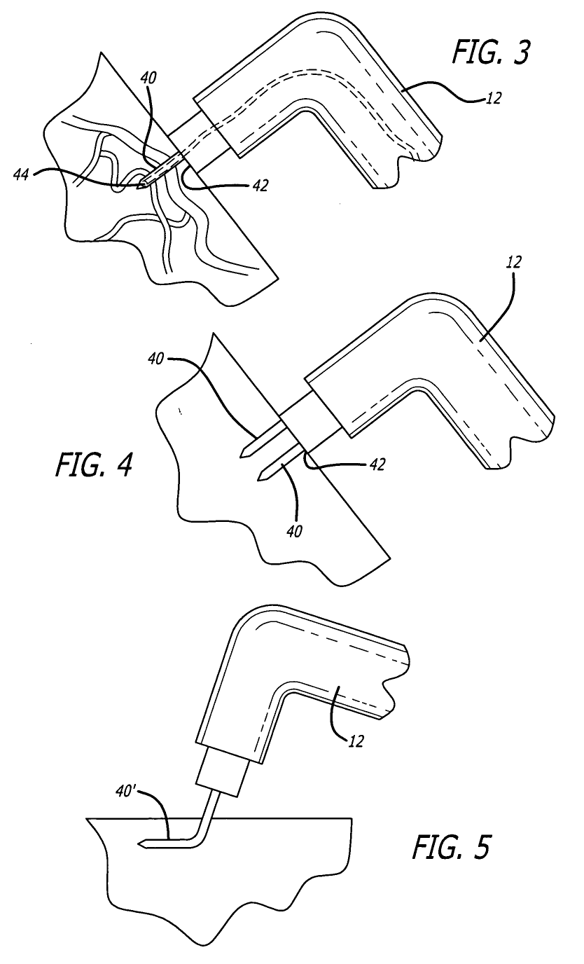

FIG. 3, shows an embodiment of a probe 12 with a tip 40 that is inserted into skin tissue. The probe 12 may have a stop 42 that limits the insertion depth of the tip 40. Although a stop 42 is shown and described, it is to be understood that the probe 12 does not need to have a stop 42. The stop 42 may be constructed from an insulator material to minimize the flow of current on the top layer of skin.

The tip 40 should have a length that insures sufficient penetration into the dermis layer of the skin. By way of example, the tip 40 may have a length between 600 to 800 microns. It has been found that a tip 600 microns long can be used to treat facial spider veins and a tip 800 microns long can be used to treat wrinkles.

The diameter of the tip 40 should be sufficient to provide the desired amount of energy but be small enough to not leave unsightly incision wounds. It has been found that a tip 90 microns in diameter can be used to treat facial spider veins and a tip diameter of 200 to 300 millimeters can be used for wrinkles. The tip 40 could carry, have embedded in it, or otherwise attached to it, specialized sensors 44, such as temperature sensors (e.g. thermocouples, thermistors, etc.), pressure sensors, etc. Although specific lengths and diameters have been disclosed, it is to be understood that the tip may have different lengths and diameters.

In operation, the treated skin is typically washed and then wiped with alcohol. The probe tip 40 is inserted into the skin and then radio frequency energy is delivered from the console to the tissue. The electrical current flows through the skin tissue and generates heat within the tissue. The heat denatures the collagen within the dermis layer of the skin. The denatured collagen shrinks the tissue. When applied adjacent to a blood carrying tissue such as a facial spider vein the shrunken tissue reduces or eliminates the visual appearance of the vein. When applied to age folds (wrinkles) the shrunken tissue reduces or eliminates the wrinkle.

The process of inserting the probe tip 40 and delivering radio frequency energy can be repeated to create a plurality of denatured areas. The denatured areas can be repeated in a pattern where the areas are separated by 2 to 3 millimeters. When treating exposed veins it is desirable to create a pattern of denatured areas that follows the path of the vein.

Following the procedure the patient may be given oral minocycline, 100 milligrams, every 12 hours for 4 days. The treated area may also be regularly washed with antiseptic soap and water.

FIG. 4 shows an alternate embodiment of a probe that has a pair of tips 40. The probe may be a bi-polar device wherein one of the tips is a return ground element. Alternatively, combined bi-polar/monopolar energy delivery could be employed. In such case, an indifferent return electrode could be used in conjunction with a bi-polar probe. The use of a bi-polar probe may create a flow of current, and resultant collagen shrinkage, that is essentially parallel with the surface of the skin. Additionally, bi-polar energy applications have the advantage of confining treatment to better-specified boundaries. Such bi-polar probes could be used in situations when the target treatment zone is limited in size.

FIG. 5 shows another embodiment of a probe with a bent tip 40′. The bent tip 40′ may be inserted into the skin in a manner so that the tip is essentially parallel with the skin surface. The probe may have a pair of bent tips 40′ so that the tips 40′ create a flow of current, and resultant collagen shrinkage, that produces a relatively large plane of shrunken collagen.

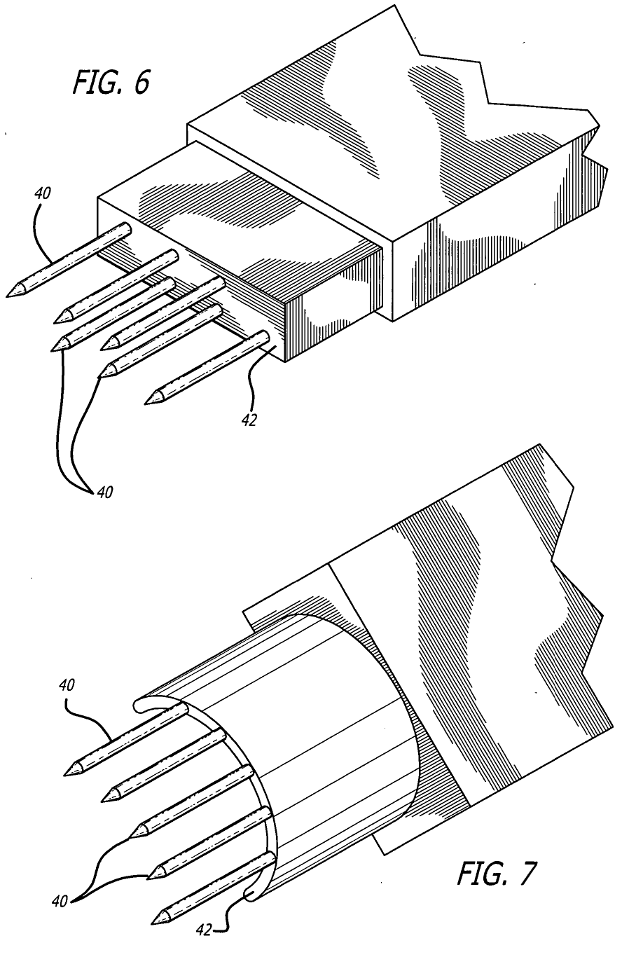

FIG. 6 shows another embodiment of a probe with a plurality of tips 40. The tips 40 may be connected to the console so that there are a number of bi-polar tip pairs. This embodiment allows for the simultaneous creation of multiple denatured areas, thereby reducing the time required to perform a procedure.

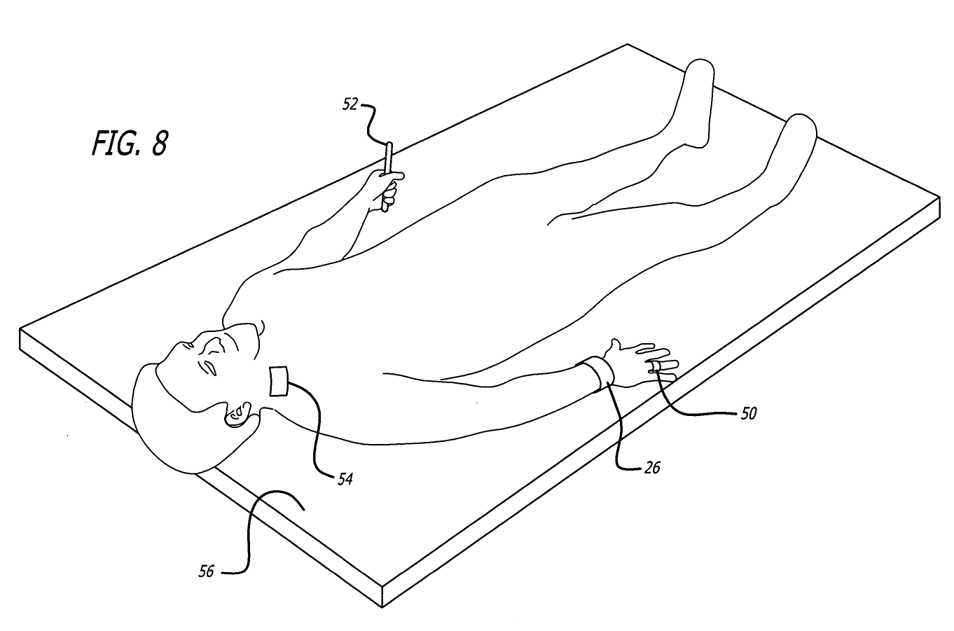

FIG. 7 shows another embodiment of a probe with a plurality of tips 40 arranged in a pattern. This probe may also allow for the simultaneous creation of multiple denatured areas to reduce the time required to perform a procedure.

FIG. 8 shows the use of different ground elements 26, 50, 52, 54 and 56. The ground element could be configured as a ring 50 worn on the patient's finger, a wristband 26 wrapped around the patient's wrist or a rod 52 held by the patient. The ground element may be configured as a patch 54 that is attached to the patient by an adhesive or a plate 56 placed under the patient. Although the plate 56 is shown as being larger than the patient, the plate 56 may have different sizes and shapes.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), microwave, ultrasonic and light can be transferred into the skin tissue through the probe.

By way of example, the console can be modified to supply energy in the microwave frequency range or the ultrasonic frequency range. By way of example, the probe may have a helical microwave antenna with a diameter suitable for delivery into the tissue. The delivery of microwave energy could be achieved with or without tissue penetration, depending on the design of the antenna. The system may modulate the microwave energy in response to changes in the characteristic impedance.

For ultrasonic application, the probe would contain a transducer that is driven by the console and mechanically oscillates the tip. The system could monitor acoustic impedance and provide a corresponding feedback/regulation scheme. For application of light the probe may contain some type of light guide that is inserted into the skin and directs light into the tissue. The console would have means to generate light, preferably a coherent light source such as a laser, that can be delivered by the probe. The probe may include lens, waveguide and a photodiode that is used sense reflected light and monitor variations in the index of refraction, birefringence index of the tissue as a way to monitor physiological changes and regulate power.

1. A method for reducing the visual appearance of blood carrying tissue, comprising:
placing a probe in contact with a skin tissue;
delivering energy into the skin tissue through the probe to denature the skin tissue and reduce the visual appearance of the blood carrying tissue.

2. The method of claim 1, wherein the placing of the probe and the delivery of energy is repeated to create a plurality of denatured areas.

3. The method of claim 2, wherein the denatured areas extend along a path of the blood carrying tissue.

4. The method of claim 2, wherein the denatured areas are approximately 2 to 3 millimeters apart.

5. The method of claim 1, wherein the probe is inserted into the skin tissue.

6. The method of claim 1, wherein the delivery of energy occurs over a predetermined time interval established by a console that provides the energy.

7. The method of claim 1, further comprising coupling a ground element to the skin tissue.

8. The method of claim 1, wherein the probe has a plurality of tips that are inserted into the skin tissue.

9. The method of claim 8, wherein the energy flows through the skin tissue between at least two tips.

10. The method of claim 1, further comprising sensing a temperature of the skin tissue.

11. The method of claim 10, wherein the energy is varied in response to the sensed temperature.

12. The method of claim 1, further comprising sensing an impedance of the skin tissue.

13. The method of claim 12, wherein the energy is varied in response to the sensed impedance.

14. The method of claim 1, wherein the energy is in a radio frequency range.

15. A probe used in a medical procedure on skin tissue, comprising:
a stop; and,
a first tip that extends from said stop.

16. The probe of claim 15, further comprising a second tip that extends from said stop.

17. The probe of claim 15, further comprising a plurality of second tips that extend from said stop.

18. The probe of claim 17, wherein said second tip provides a return path.

19. The probe of claim 15, wherein said stop is constructed from an insulator material.

20. The probe of claim 15, wherein said first tip has a diameter greater than 90 microns.

21. The probe of claim 15, further comprising a temperature sensor coupled to said first tip.

22. An apparatus used in a medical procedure on skin tissue, comprising:
a probe that has a stop, and a first tip that extends from said stop; and,
a console that delivers energy to said probe.

23. The apparatus of claim 22, further comprising a second tip that extends from said stop.

24. The apparatus of claim 22, further comprising a plurality of second tips that extend from said stop.

25. The apparatus of claim 23, wherein said second tip provides a return path.

26. The apparatus of claim 22, wherein said stop is constructed from an insulator material.

27. The probe of claim 22, wherein said first tip has a diameter greater than 90 microns.

28. The apparatus of claim 22, wherein said console delivers energy to said probe for a predetermined time interval established by said console.

29. The apparatus of claim 22, further comprising a ground element coupled to said console.

30. The apparatus of claim 22, wherein the energy is in a radio frequency range.

31. The apparatus of claim 22, wherein the energy is in a microwave frequency range.

32. The apparatus of claim 22, wherein the energy is ultrasonic.

33. The apparatus of claim 22, wherein the energy is light.

34. The apparatus of claim 22, wherein said probe includes a temperature sensor.

35. The apparatus of claim 22, wherein said console delivers energy to the skin tissue in a manner to maintain a temperature of the skin tissue at a certain value.

36. The apparatus of claim 22, wherein said console delivers energy and senses a tissue impedance, said console decreases the energy if the impedance increases.

37. The apparatus of claim 22, wherein said console delivers energy for a time interval modulated based on a physiological parameter.

38. The apparatus of claim 22, wherein the physiological parameter is tissue impedance.

39. An apparatus used in a medical procedure on skin tissue, comprising:
a console coupled to said probe; and,
probe means for delivering energy from said console to the skin tissue.

40. The apparatus of claim 39, wherein said probe means includes a stop, and a first tip that extends from said stop.

41. The apparatus of claim 40, wherein said probe means includes a second tip that extends from said stop.

42. The apparatus of claim 40, wherein said probe means includes a plurality of second tips that extend from said stop.

43. The apparatus of claim 41, wherein said second tip provides a return path.

44. The apparatus of claim 40, wherein said stop is constructed from an insulator material.

45. The apparatus of claim 40, wherein said first tip has a diameter greater than 90 microns.

46. The apparatus of claim 39, wherein said console delivers energy to said probe for a predetermined time interval established by said console.

47. The apparatus of claim 39, further comprising a ground element coupled to said console.

48. The apparatus of claim 39, wherein the energy is in a radio frequency range.

49. The apparatus of claim 39, wherein the energy is in a microwave frequency range.

50. The apparatus of claim 39, wherein the energy is ultrasonic.

51. The apparatus of claim 39, wherein the energy is light.

52. The apparatus of claim 39, wherein said probe includes a temperature sensor.

53. The apparatus of claim 39, wherein said console delivers energy to the skin tissue in a manner to maintain a temperature of the skin tissue at a certain value.

54. The apparatus of claim 39, wherein said console delivers energy and senses a tissue impedance, said console decreases the energy if the impedance increases.

55. The apparatus of claim 39, wherein said console delivers energy for a time interval modulated based on a physiological parameter.

56. The apparatus of claim 39, wherein the physiological parameter is tissue impedance.
